(19) United States
(12) Patent Application Publication
(10) Pub. No.: US 2006/0013772 A1
(43) Pub. Date: Jan. 19, 2006

(54) Method and device to recover diagnostic and therapeutic agents

(75) Inventors: Martin M. LeWinter, Williston, VT (US); Harold L. Dauerman, Shelburne, VT (US); Daniel L. Lustgarten, Burlington, VT (US); Markus Meyer, Burlington, VT (US)

Correspondence Address: Wolf, Greenfield & Sacks, P.C.;Federal Reserve Plaza, 600 Atlantic Avenue, Boston, MA, 02210-2206, US

(73) Assignee: University of Vermont and State Agricultural College, Burlington, VT (US)

(21) Appl. No.: 11/169,516

(22) Filed: Jun. 29, 2005

Related U.S. Application Data
(60) Provisional application No. 60/584,238, filed on Jun. 30, 2004.
(60) Provisional application No. 60/668,402, filed on Apr. 5, 2005.

(51) Int. Cl. A61K 48/00; A61M 31/00

(52) U.S. Cl. 424/9.52; 424/93.2; 604/500

(57) ABSTRACT

The invention relates to a method and device for recovering therapeutic and diagnostic agents from the venous outflow of a tissue or organ. A method for recovering an agent includes inserting a catheter into a vascular outflow of a tissue or organ, occluding the flow of blood/fluid through the vascular outflow, and withdrawing blood/fluid in the vascular outflow into the catheter to recover at least a portion of the agent administered to the vascular inflow of the organ or tissue. Also provided is a device to capture an agent administered to a vascular inflow, the device having a catheter having an expandable occlusion device to occlude a flow of fluid through the vascular outflow, a pump operably coupled to the catheter for withdrawing from the vascular outflow at least a portion of the agent administered to a vascular inflow of the organ or tissue, and an automated controller in signal communication with the catheter to control the expandable occlusion device and the pump to withdraw at least a portion of the agent from the vascular outflow.

### RELATED APPLICATIONS

This application claims benefit under 35 U.S.C. 119(e) of U.S. Provisional Patent Application Ser. Nos. 60/584,238, filed Jun. 30, 2004, and 60/668,402, filed Apr. 5, 2005, the entire contents of which are incorporated herein by reference.

### BACKGROUND OF INVENTION

1. Field of Invention

This invention relates to a method for recovering therapeutic and diagnostic agents during medical procedures, and a device for recovering such agents.

2. Description of Related Art

The use of diagnostic or therapeutic agents to facilitate the examination or treatment of internal organs and tissues is common medical practice. A diagnostic agent or dye may be injected into the blood flow serving an organ or tissue as part of a radiographic procedure, such as an x-ray, performed to help diagnose any problems or abnormalities with the organ or tissue. A therapeutic agent may be introduced into the blood flow serving a tissue or an organ to improve the medical condition of a patient.

Coronary angiography, percutaneous coronary intervention, and cardiac catheterization are exemplary procedures that commonly use a radiographic contrast agent or medium to facilitate a diagnosis or therapeutic intervention. However, it has become common knowledge to those of skill in the art that the use of intravascular contrast agents is associated with an increased risk of contrast-induced acute renal failure (CIARF), which may lead to excess rates of morbidity and mortality. The potential consequences of CIARF range from transient elevations of serum creatinine concentration, an indicator of renal dysfunction, to end stage renal failure requiring permanent renal function replacement therapy—and even death. Those patients most susceptible to CIARF include those with pre-existing renal disease, i.e., the lower the creatinine clearance prior to contrast exposure, the greater the probability of CIARF. Additionally, patients with diabetes mellitus and conditions that predispose to development of ARF, e.g., sepsis, are also at increased risk of CIARF.

In order to prevent or at least reduce the risk of CIARF, a number of strategies have been attempted, such as volume expansion (sometimes referred to as hydration), decreasing the amount of contrast agent used in the patient, prophylactic dialysis, administering iso-osmolar contrast agents or the antioxidant acetylcysteine during catheterization, and continuous venovenous hemofiltration before and after contrast agent introduction. However, these strategies have significant shortcomings with respect to efficacy, they require increased drug usage and/or prolonged hospitalization (both of which increase treatment costs), and, in the case of decreasing the amount of contrast agent used during the procedure, a lessened ability to adequately visualize the vasculature.

### SUMMARY OF THE INVENTION

The invention provides methods and devices useful for recovering at least a portion of an agent that has been injected into a blood vessel serving an organ or tissue in the body. In this manner, the devices and methods of the present invention decrease the amount of the agent that leaves the organ or tissue and enters into the rest of the circulatory system.

In one aspect, the invention provides a method for recovering an agent introduced into a vascular inflow of an organ or tissue. The method according to this aspect includes the steps of inserting a distal end of a catheter into a vascular outflow of an organ or tissue, the catheter comprising a lumen, an outer surface, and an expandable occlusion device operably coupled to the outer surface at the distal end to occlude a flow of fluid through the vascular outflow while the lumen is in fluid communication with fluid in the vascular outflow; occluding the flow of fluid through the vascular outflow by expanding the occlusion device; and withdrawing or passively draining fluid in the vascular outflow through the lumen to recover at least a portion of an agent administered to a vascular inflow of the organ or tissue.

In one aspect, the invention provides a method of recovering contrast agent during coronary angiography. The method according to this aspect includes the steps of inserting a catheter into a coronary sinus, the catheter comprising a lumen, an outer surface, a proximal end and a distal end, and an expandable occlusion device operably coupled to the outer surface of the lumen at the distal end to occlude a flow of fluid through a coronary sinus while the lumen is in fluid communication with fluid in through the coronary sinus; introducing a contrast agent to a coronary artery; expanding the occlusion device to occlude the flow of fluid through the coronary sinus; and withdrawing fluid from the coronary sinus into the lumen to recover at least a portion of the contrast agent introduced into the coronary artery.

In one aspect, the invention provides an apparatus to capture an agent introduced into a vascular inflow of an organ or tissue. The apparatus according to this aspect includes a catheter having a lumen, an outer surface, a proximal end and a distal end, the distal end being insertable into a vascular outflow of an organ or tissue, and an expandable occlusion device operably coupled to the outer surface at the distal end to occlude a flow of fluid through the vascular outflow; a pump, operably coupled to the catheter, configured and arranged to withdraw fluid from the vascular outflow into the lumen; and an automated controller in signal communication with the catheter to control the expandable occlusion device to occlude the flow of fluid through the vascular outflow, and to control the pump to withdraw at least a portion of the agent from the vascular outflow, the controlling of the occlusion device and the pump based on detection of the presence of the agent introduced into the vascular inflow.

In one aspect, the invention provides a device to capture a contrast agent introduced into a coronary artery during a coronary angiography. The apparatus according to this aspect includes a catheter having a lumen, an outer surface, a proximal end and a distal end, the distal end being insertable into a coronary sinus, and an inflatable balloon operably coupled to the outer surface at the distal end to occlude a flow of fluid in the coronary sinus; a pump, operably coupled to the catheter, configured and arranged to withdraw fluid from the coronary sinus into the lumen; and an automated controller in signal communication with the catheter to control the inflatable balloon to occlude the flow of fluid in the coronary sinus, and to control the pump to withdraw at least a portion of the agent from the vascular outflow, the controlling of the occlusion device and the pump based on timing and rate of introduction of an agent introduced into the vascular inflow.

### BRIEF DESCRIPTION OF THE DRAWINGS

Aspects of the invention are described in connection with the following illustrative drawings in which like numerals reference like elements, and wherein:

FIG. 1 is a schematic diagram of an embodiment of the present invention;

FIG. 2 is a flow chart showing a method of the present invention; and

FIG. 3 is a graphical and pictorial representation of exemplary results using an embodiment of the present invention.

### DETAILED DESCRIPTION

This invention is directed to a device and method useful for recovering a therapeutic or diagnostic agent from the vascular outflow of an organ or tissue following introduction of the agent into a vascular inflow of the organ or tissue. For purposes herein, vascular outflow means a vascular structure through which blood drains from an organ or tissue. Likewise, vascular inflow means a vascular structure through which blood flows into an organ or tissue. It should be understood that this invention is not limited in its application to the specific embodiments and details set forth in the following description and illustrated in the accompanying drawings. The invention is capable of other embodiments and of being practiced or of being carried out in various ways. Also, the phraseology and terminology used herein is for the purpose of description and should not be regarded as limiting. The use of “including”, “comprising”, or “having”, “containing”, “involving”, and variations thereof herein, is meant to encompass the items listed thereafter and equivalents thereof as well as additional items.

The present invention provides a novel method and device for removing fluid (e.g., blood) that includes at least a portion of an agent that has been inserted or otherwise introduced into the vasculature of an organ or tissue for diagnostic or therapeutic purposes. The invention can have application in conjunction with coronary procedures or in procedures involving other organs such as, but not limited to, for example, kidney, brain, lung, and liver. The invention can also be used in conjunction with medical procedures involving the vasculature of bodily tissue not part of an internal organ, such as in an upper or lower extremity.

The agent that can be recovered in accordance with the present invention can be a diagnostic agent, a therapeutic agent, or a combination diagnostic and therapeutic agent. The agent can be a noxious or non-noxious substance, and may be radiopaque for use with, e.g., x-ray examinations, or not radiopaque for use with, e.g., magnetic resonance imaging examinations. A diagnostic agent can be a contrast medium or agent, by which is meant a substance that is introduced into or around tissue or an organ and allows radiographic visualization of the tissue or organ. In one embodiment the contrast agent or medium is an intravascular contrast agent. Examples of contrast agents include Isovue, Iohexol, loxaglate, Iodixanol, and other iodinated contrast materials. A diagnostic agent as described above can be used in exemplary radiographic procedures including both coronary and non-coronary angiographies, such as coronary angiograms, aortograms, ventriculograms, limb angiograms and peripheral angiographies, renal artery studies, cerebral circulation studies, pulmonary angiograms, coronary sinus venograms, as well as other applications in organs such as a kidney, lung, liver, or brain, or in bodily tissue with sufficient venous flow to withdraw an agent introduced into the fluid flow. Procedures involving any organs or tissues in which the veins are accessible by catheter thereby allowing recovery of an agent introduced into the vasculature supplying the organ or tissue can potentially benefit from the present invention. The invention can also be used to recover therapeutic agents that may be toxic if they enter the general circulation, for example, drugs, chemotherapeutic agents, viral agents delivered as vectors for gene therapy or cell transfer. Additionally, occlusion of the venous drainage by inflation of the balloon during delivery of such drugs or agents may allow for a longer period of exposure of the organ or tissue to the drug or agent with resultant improved therapeutic efficacy.

Gene therapy is a method of correcting functional gene loss by delivering genes to human or other tissues, or simply expression of a therapeutic gene in a target tissue, to achieve benefit. For delivery to cells, a functional copy of the therapeutic gene is incorporated into a nucleic acid vector and placed under control of one or more regulatory elements which permit expression of the gene when introduced into a target cell to be treated. This type of therapy can be performed ex vivo or, more commonly, in vivo. In ex vivo gene therapy, cells are removed from a subject, treated outside of the subject, and then returned to the subject. See U.S. Pat. No. 5,399,346. In contrast, in vivo gene therapy involves administration of the therapeutic gene itself, in vivo. Gene therapy can be performed using either low efficiency non-viral techniques (e.g., liposome- or gene gun-mediated gene transfer) or, more commonly, high efficiency viral infection techniques. Virally based in vivo gene therapy has been promoted and is undergoing clinical studies for use in humans for the treatment of a number of diseases, including heart diseases such as congestive heart failure and myocardial ischemia.

Often DNA viruses engineered to be safe or nonviral DNA are used to help deliver a healthy gene to cells of the target tissue. In general, non-viral vectors are very inefficient at delivering genes to cardiovascular cells. Viruses are more efficient but also have a higher safety risk. Genetic modification of genomes from different virus families (Adenoviridae, Retroviridae, Herpesviridae) has resulted in the development of gene therapy vectors with a similar capacity to infect cells or tissues as that of wild-type viruses. In contrast to wild-type viruses, gene therapy vectors are engineered to transfer therapeutic genes into the target cells or tissues. In addition, they have been engineered to reduce or eliminate their capacity for replication in target cells, through the removal of essential genes.

In certain embodiments, a virus vector for delivering a nucleic acid molecule encoding a protein of interest is selected from the group consisting of adenoviruses, adeno-associated viruses, poxviruses including vaccinia viruses and attenuated poxviruses, Semliki Forest virus, Venezuelan equine encephalitis virus, retroviruses, Sindbis virus, and Ty virus-like particle. Examples of viruses and virus-like particles which have been used to deliver exogenous nucleic acids include: replication-defective adenoviruses (e.g., Xiang et al., Virology 219:220-227, 1996; Eloit et al., J. Virol. 7:5375-5381, 1997; Chengalvala et al., Vaccine 15:335-339, 1997), a modified retrovirus (Townsend et al., J. Virol. 71:3365-3374, 1997), a nonreplicating retrovirus (Irwin et al., J. Virol. 68:5036-5044, 1994), a replication-defective Semliki Forest virus (Zhao et al., Proc. Natl. Acad. Sci. USA 92:3009-3013, 1995), canarypox virus and highly attenuated vaccinia virus derivative (Paoletti, Proc. Natl. Acad Sci. USA 93:11349-11353, 1996), non-replicative vaccinia virus (Moss, Proc. Natl. Acad. Sci. USA 93:11341-11348, 1996), replicative vaccinia virus (Moss, Dev. Biol. Stand. 82:55-63, 1994), Venezuelan equine encephalitis virus (Davis et al., J. Virol. 70:3781-3787, 1996), Sindbis virus (Pugachev et al., Virology 212:587-594, 1995), and Ty virus-like particle (Allsopp et al., Eur. J. Immunol 26:1951-1959, 1996). In preferred embodiments, the virus vector is an adenovirus.

Another preferred virus for certain applications is the adeno-associated virus (AAV, including both wild-type and recombinant forms of AAV), a double-stranded DNA virus. AAV is capable of infecting a wide range of cell types and species and can be engineered to be replication-deficient. It further has advantages, such as heat and lipid solvent stability, high transduction frequencies in cells of diverse lineages, including hematopoietic cells, and lack of superinfection inhibition thus allowing multiple series of transductions. AAV can integrate into human cellular DNA in a site-specific manner, thereby minimizing the possibility of insertional mutagenesis and variability of inserted gene expression. In addition, wild-type AAV infections have been followed in tissue culture for greater than 100 passages in the absence of selective pressure, implying that the AAV genomic integration is a relatively stable event. AAV can also function in an extrachromosomal fashion. AAV is currently considered by many to be the vector of choice for in vivo gene therapy in humans.

In general, other preferred viral vectors are based on non-cytopathic eukaryotic viruses in which non-essential genes have been replaced with the gene of interest. Non-cytopathic viruses include retroviruses, the life cycle of which involves reverse transcription of genomic viral RNA into DNA with subsequent proviral integration into host cellular DNA. Adenoviruses and retroviruses have been approved for human gene therapy trials. In general, the retroviruses are replication-deficient (i.e., capable of directing synthesis of the desired proteins, but incapable of manufacturing an infectious particle). Such genetically altered retroviral expression vectors have general utility for the high-efficiency transduction of genes in vivo. Standard protocols for producing replication-deficient retroviruses (including the steps of incorporation of exogenous genetic material into a plasmid, transfection of a packaging cell lined with plasmid, production of recombinant retroviruses by the packaging cell line, collection of viral particles from tissue culture media, and infection of the target cells with viral particles) are provided in Kriegler, M., Gene Transfer and Expression, A Laboratory Manual, W. H. Freeman Co., New York (1990) and Murry, E. J., Ed., Methods in Molecular Biology, vol. 7, Humana Press, Inc., Cliffton, N.J. (1991).

Preferably the foregoing nucleic acid delivery vectors: (1) contain exogenous genetic material that can be transcribed and translated in a mammalian cell, and (2) contain on a surface a ligand that selectively binds to a receptor on the surface of a target cell, such as a mammalian cell, so as to facilitate entry of the therapeutic gene into the target cell.

A number of therapeutic genes have been studied in preclinical and clinical studies of gene therapy for the treatment of heart disease and other forms of vascular disease. These genes include vascular endothelial growth factor (e.g., VEGF-1 and VEGF-2), fibroblast growth factor (e.g., FGF4), β₂ adrenergic receptor, inhibitor of β-adrenergic receptor kinase, S100A1, and heme-oxygenase 1, among others.

These gene therapy approaches to heart disease, as well as other types of disease, have been complicated by two major factors. One factor is difficulty delivering an effective amount of gene therapy to the intended target tissue. For several years in vivo gene therapy has been carried out using multiple doses of the therapeutic gene in order to achieve therapeutic efficacy. In gene therapy directed to the heart, repeated injections directly into the heart have been necessary, attended by high morbidity associated with such invasive treatment. In another approach, cardiac gene delivery is performed during cardiopulmonary bypass during open-heart surgery. Following median stemotomy and placement on cardiopulmonary bypass, adenoviral vectors containing therapeutic genes were administered to pigs through the (intracoronary) cardioplegia cannula immediately after arrest and then allowed to dwell in the coronary circulation during the thirty-minute period of aortic cross-clamping. On sacrifice one week later, treated animals had four-fold overexpression of therapeutic gene in the heart compared to untreated control animals, whereas transgene expression in lung and liver was undetectable. Davidson M J et al. (2001) Circulation 104:131-3.

Another factor that has complicated gene therapy approaches to heart disease, as well as other types of disease, is vector-associated toxicity. Adenoviral vectors, in particular but not exclusively, have been reported to activate both innate and adaptive immune responses, resulting in sometimes severe and even fatal inflammatory responses. The risk of encountering such side effects is amplified by repeated administration and high. dose administration previously necessary to achieve the desired therapeutic effect.

Thus in certain aspects the invention provides methods and devices suited for use in connection with gene therapy, particularly for the treatment of heart disease. The invention allows for effective and relatively noninvasive delivery of gene therapy agents to a heart, both by isolating the coronary circulation from the general circulation and by permitting a relatively prolonged period for contact between the agent and the target tissue. More specifically, compared to simple intracoronary administration, occlusion of the venous drainage by inflation of the balloon during delivery of such drugs or agents allows for a longer period of exposure of the heart tissue to the drug or agent with resultant improved therapeutic efficacy. In addition, using the methods and devices of the invention, removal of the drug or agent from the coronary circulation via an occlusive catheter positioned in the coronary sinus dramatically reduces risks of toxicity and exposure of noncardiac tissues to the gene therapy drug or agent.

Enhanced delivery of a gene therapy agent to noncardiac target organs or tissues, along with reduced risks of toxicity and exposure of other tissues to the gene therapy drug or agent can similarly be accomplished using the methods and devices of the invention described herein.

Furthermore, by impeding venous clearance there is more time for any therapeutic agent, not just gene therapy agents, to contact target cells and saturate receptor targets. This may lead to improved uptake of any given therapeutic agent by target cells, thereby enhancing therapeutic efficacy of any agent administered using the methods and devices of the invention.

In one embodiment, the agent to be recovered is introduced into a vascular inflow of an organ or tissue. In one embodiment, the agent is introduced into a coronary artery. An agent is introduced into a vascular inflow or into a coronary artery when it is introduced into the lumen of the vascular structure (i.e., vascular inflow or coronary artery) such that the agent is introduced into fluid within the vascular inflow or the coronary artery.

In one embodiment of the present invention, a distal end of a catheter of the invention, also referred to herein as a capture catheter, is inserted into a vascular outflow of an organ or tissue to withdraw blood and at least a portion of an agent from the organ or tissue. The agent can be introduced into the vascular inflow of the organ or tissue, e.g., by another catheter (insertion catheter). The vascular outflow can include a vein draining the organ or tissue while the vascular inflow can include an artery supplying the organ or tissue. After the agent is introduced into the vascular inflow by an insertion catheter and flows through the vascular system of the organ or tissue, a catheter can be in place to capture at least some of the agent before it exits the organ or tissue to enter the general circulatory system. To do this, the capture catheter can first occlude the flow of fluid containing a mixture of blood and the agent in the vascular outflow and then withdraw at least some of the fluid through a lumen of the catheter that is in fluid communication with fluid in the vascular outflow.

The capture catheter can be that which is used in any of multiple applications, coronary and non-coronary. It can be a manually or automatically controlled catheter, configured in any of multiple ways, and can, in one embodiment, be the type of catheter used in “open atrium” cardiac procedures (e.g., a catheter directly accessing the coronary sinus) or “blind” cardiac procedures (e.g., a catheter introduced into the right atrium and steered into the coronary sinus), or other procedures and techniques, not limited to cardiac applications, known to those of ordinary skill in the art.

The capture catheter can be of various sizes, so long as its distal end can be inserted into the venous outflow for use according to a method of the invention. The catheter can be made of any of numerous materials, including silicone rubber, nylon, polyurethane, polyethylene, polyvinyl chloride, optionally with Teflon® or steel braid or other support materials, or other materials known to those in the art. The catheter typically will include at least a flow lumen for the flow of fluid into the catheter. The flow lumen can have an inside diameter of various sizes within the full range of vascular diameters (i.e., from approximately 10 micrometers to 4 centimeters). The catheter can also include an inflation lumen to inflate or deploy an occlusion device (discussed further below). The catheter in some embodiments may include one or more additional lumens, e.g., a pressure lumen to detect the amount of suction force or pressure exerted by the catheter. In one embodiment, the catheter can be wire-reinforced. The proximal end of the catheter can include, or have connections for, a pump, syringe or other fluid withdrawal means, as well as a controller or one or more sensors to facilitate the fluid withdrawal. The distal end of the catheter can include an occlusion device to occlude the flow of fluid beyond the catheter, and optionally one or more sensors, electrodes, or leads to assist in detecting the presence of an agent in the vascular outflow.

In one embodiment, the catheter has an expandable occlusion device at the distal end of the catheter. As used herein, “at the distal end of the catheter” refers to a location encompassing or substantially adjacent to the distal end of the catheter.

As mentioned, the capture catheter can include an occlusion device to occlude a flow of fluid from the organ or tissue. In one embodiment, the occlusion device can be an inflatable balloon or other expandable member such as an umbrella-like device that surrounds and is secured to the outer surface of the catheter tube near the distal end of the catheter. The occlusion device can be integral to the outer surface or it can be secured to the outer surface by any number of means, such as by molding, bonding, welding, mounted by mechanical fasteners, as well as by other means known to those of skill in the art. In the embodiment where a balloon is used for occlusion purposes, the balloon can be made of various materials of appropriate elasticity, for example, natural or synthetic rubber, polyethylene, polyethylene teraphalate, polyolyfin, as well as other elastomers known to those of ordinary skill in the art. The catheter or at least the balloon may be coated with friction-resistant coatings (such as silicone or polyethylene oxide). The balloon can be shaped in various forms in its inflated state, such as spherical, tubular, acorn-like, to name a few. The balloon can be sized according to the size of the bodily region with which it will function. An exemplary range of sizes for a balloon of the present invention in an inflated state can be from less than a millimeter to four centimeters.

The balloon can be inflated and deflated manually or automatically by any of various mechanisms that is either part of the catheter or connected thereto. For example, the catheter can include an inflation lumen in fluid communication with the balloon to transfer fluid to the balloon, causing it to inflate. The fluid can be, for example, water, saline solution, air, or any contrast or diagnostic agent that is under pressure. In one embodiment, the balloon can be inflated manually (e.g., by an integral or attachable manual syringe). In another embodiment, a pump or other mechanical or electromechanical device can receive a signal input from a controller to cause the balloon to inflate based on an electrical signal.

In yet another embodiment, the occlusion can be performed without the use of an expandable component of the catheter, such as by using a catheter with a distal end of such a shape that simply by inserting the catheter into the vascular outflow of the tissue or an organ, the outflow would be occluded, or if by advancing the catheter so far that its tip itself occludes the venous outflow as it enters smaller diameter vessels/veins. For example, a catheter having a wide conical shaped distal end can accomplish the same intended occlusion function. It should be understood that other configurations for the occlusion of fluid, beyond those embodiments specifically described herein but known to those of ordinary skill in the art, are also within the scope of the invention.

To withdraw the fluid into the capture catheter, the catheter can be designed to apply negative pressure to the fluid in the outflow portion of the organ or tissue to withdraw fluid into the catheter. This withdrawal function can be accomplished through various means, such as by using a pump integral with or connected to the catheter, and configured and arranged to create negative pressure (i.e., suction) through a flow lumen of the catheter. When employed in an organ or tissue, the catheter, and specifically the distal end, can be in contact with the vascular outflow of the organ or tissue such that the flow lumen is in fluid communication with fluid in the vascular outflow to facilitate the withdrawal of fluid through the lumen toward the proximal end of the catheter. The pump can be actuated by a motor internal or external to the pump, or can be actuated by other means such as, for example, by hydraulics. In one embodiment, the fluid withdrawal from the vascular outflow recovers at least 50 percent of the agent introduced into the vascular inflow.

The withdrawal force can also be accomplished by other means besides a pump separate from or integral with the capture catheter. For example, the catheter can include a component that acts similar to a syringe that, when actuated, creates negative pressure in the flow lumen to draw fluid into the lumen. In another embodiment, the syringe-like device can be separate from the catheter and attachable to, for example, a port in the catheter to effect the withdrawal force. In yet another embodiment, passive emptying, taking advantage of the higher pressure in the venous circulation compared with ambient atmospheric pressure, can be used to withdraw fluid. The invention, and more specifically the means to withdraw fluid into the catheter, is not limited to the pump and syringe embodiments described above. Other withdrawal means known to those of ordinary skill in the art are also within the scope of the invention.

The present invention can also include one or more controllers that can be automated to control the rate, timing, or both rate and timing of any of the functions described above, including but not limited to the rate and timing of a withdrawal of an agent into a flow lumen, as well as the expansion of an occluding device. It should be understood that any reference herein relative to a controller is intended to include embodiments where more than one controller exists to carry out the control function. In one embodiment, the controller is an automated controller. The controller can include one or more components of a processor, microprocessor, or a computer system that performs any of the functions described herein. The controller can also include a solenoid device to control actuation of one or more aspects (e.g., a syringe) of the catheter. The controller can be implemented in any of numerous ways, such as with dedicated hardware or using a processor that is programmed using microcode or software to perform any of the functions described herein. The controller can also include pump or occlusion device actuators, as well as visual indicators of such things as the elapsed time of balloon inflation, withdrawal force, or rate of flow of the withdrawn fluid.

The controller can be configured to receive input electronic signal communication from one or more sources and to transmit such electronic signal communication to perform any of the functions described herein. For example, in one embodiment of the present invention, a controller can be in signal communication with an insertion catheter that can be used to introduce an agent into the vascular inflow of an organ or tissue. The controller can receive signal input from an insertion catheter, or a controller or other component connected to the insertion catheter, that can include the timing of the agent introduced and/or the rate of insertion into the vascular inflow.

The controller can also receive signal input from one or more sensors that detect the presence of an agent in the vascular outflow of the organ or tissue. A sensor can be any device that senses a stimulus (e.g., light, sound, pressure, chemical, conductivity, electromagnetism) and generates a signal in response to the stimulus. The sensor can be integrated with or secured to the capture catheter in proximity to the distal end of the catheter tube or lumen such that it is insertable into a vascular outflow to detect the presence of an agent in the vascular outflow. In one embodiment, a sensor can be a conductivity sensor that senses a change in the conductivity of blood in the vascular outflow of the tissue or organ, which can be indicative of the presence of contrast medium in the blood within the organ or tissue outflow. In one embodiment, the sensor or a portion thereof can be remote from the capture catheter, or at least the distal end of the catheter, and in signal communication with one or more leads or electrodes integral with the catheter and disposed near the distal end. The leads or electrodes can detect the presence of an agent in the vascular outflow and transmit an electrical impulse or signal to the sensor, which in turn can transmit a resulting signal to a controller.

The controller can also transmit electronic signal communication to one or more components of the invention to cause a component to perform a particular function. For example, in one embodiment of the invention a controller is configured to transmit an electronic signal to a capture catheter to expand an occlusion device to block the flow of fluid when the distal end of the capture catheter is inserted into or brought into contact with the vascular outflow. In another example, a controller can transmit an electronic signal to the capture catheter that activates a withdrawal force in the capture catheter to remove at least a portion of the fluid from the vascular outflow of the organ or tissue. Either or both of these occlusion and withdrawing functions can be controlled based on input received by the controller regarding the timing and/or rate of introduction of an agent into the vascular inflow of the organ or tissue, input received by the controller regarding the presence of an agent in the vascular outflow of the organ or tissue, or other controller input known to those of ordinary skill in the art. Furthermore, it should be understood that the controller of the present invention is not limited to a particular configuration described herein or limited to the number and/or types of functions that can be controlled within the present invention.

In one embodiment, the present invention can be applicable to coronary procedures, such as cardiac catheterization and coronary angiography. For example, a patient with known or suspected coronary artery disease can undergo a coronary angiography procedure or a percutaneous coronary intervention, such as angioplasty or the implanting of a stent, any of which procedures can be combined with and benefit from the present invention. In a coronary angiography, where visualization of blood vessels of the heart is possible after injection of a radiopaque substance into a blood vessel supplying the heart, the present invention can be incorporated to withdraw at least a portion of the radiopaque substance from the heart before the substance enters the general circulation. As previously mentioned, the present invention can also be used in procedures involving organs or tissues into which an agent can be delivered and drained by way of a venous system.

In a typical coronary angiography, an agent in the form of a diagnostic contrast medium is inserted into at least one of the coronary arteries supplying the heart. As the contrast medium flows through the cardiac vascular bed, the coronary arteries can be visualized by various radiographic means. The contrast medium then flows into a cardiac vein of the heart, and then into the coronary sinus, before draining into the right atrium and eventually out of the heart into the general circulation of the body. This unrestricted flow of contrast medium into the general circulatory system is what leads to many of the adverse medical consequences described previously herein.

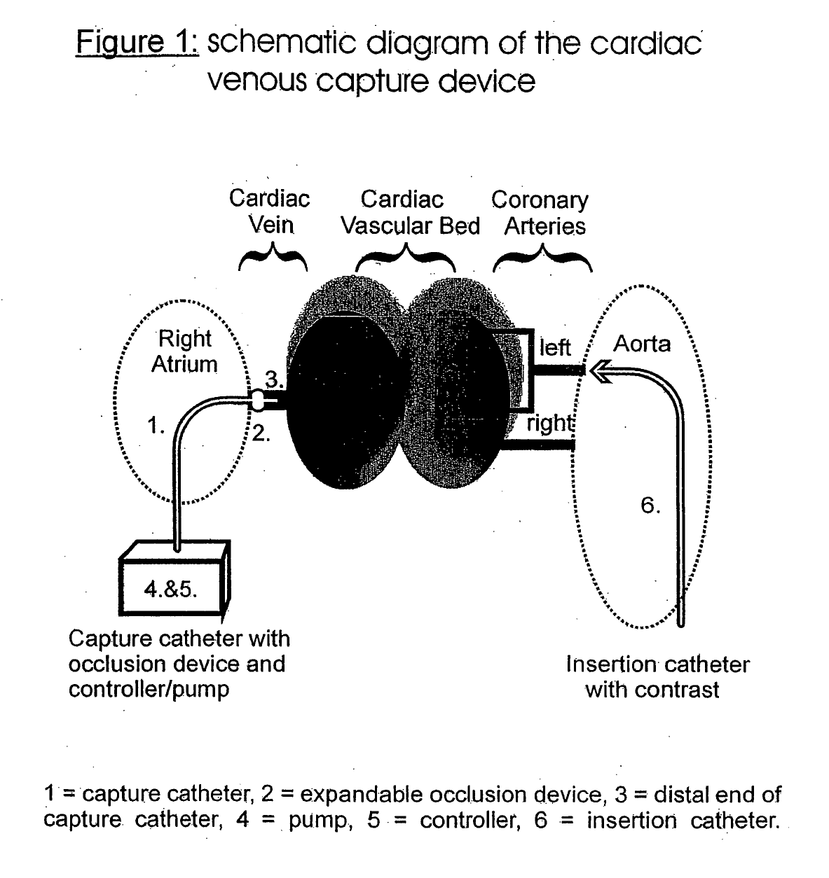

In the present invention, as depicted schematically in FIG. 1, a capture catheter 1 can be inserted through the right atrium and into the coronary sinus to withdraw at least some of the blood and contrast medium within the coronary sinus. To facilitate such contrast removal, the capture catheter 1 can include an inflatable balloon (or other expandable occlusion device) 2 at or near the distal end 3 of the catheter 1 to occlude the coronary sinus in preparation for contrast medium withdrawal. The occlusion device 2 can be disposed on and connected to an outer surface of the catheter 1, and can be inflated or otherwise expanded to prevent the flow of blood and contrast medium out of the coronary sinus and into the general circulation.

Once the capture catheter 1 is in place within the coronary sinus and the coronary sinus is occluded by the occlusion device 2, a user can then cause a lower pressure to be exerted on the blood and contrast medium within the coronary sinus to withdraw at least some of the contrast medium and blood from the coronary sinus into the catheter. This negative pressure can be created by a pump 4 either integral with or connected to the capture catheter 1. The withdrawal of contrast medium or other agents can be automatically controlled by one or more controllers 5 to coordinate the timing of the occlusion of the coronary sinus and the withdrawal of contrast medium with the timing of introduction and/or rate of introduction of the contrast medium from an insertion catheter 6 into the coronary artery. In one embodiment, introduction of the contrast agent or medium into the coronary artery can trigger the expanding of the occlusion device prior to withdrawing the blood and contrast medium from the coronary sinus. In one embodiment, the coronary sinus can be monitored (e.g., by fluoroscopy) for inflow of contrast medium prior to withdrawing blood and contrast medium from the coronary sinus.

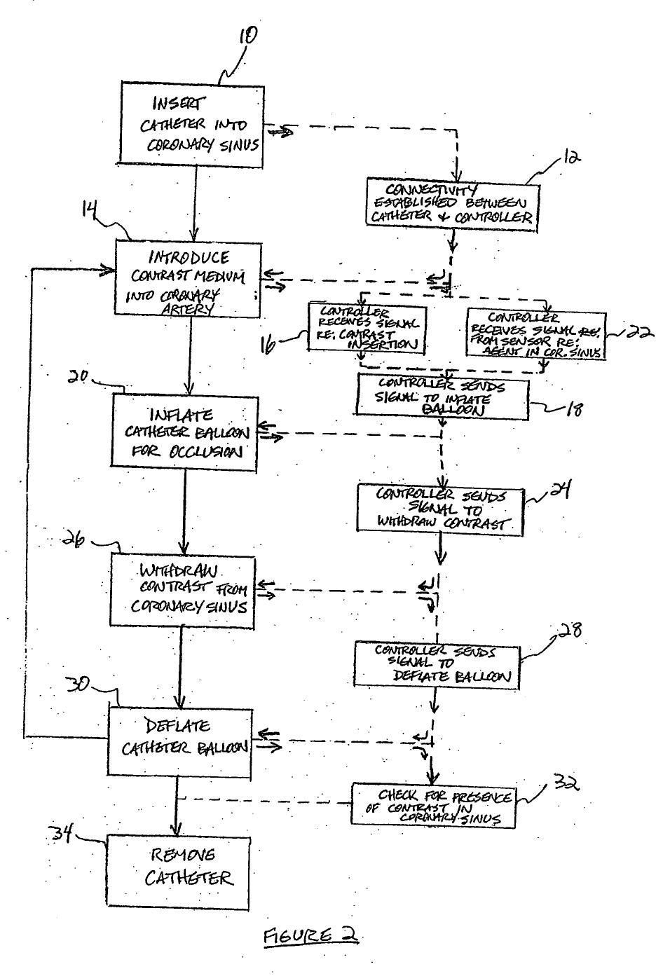

One exemplary process for removing contrast medium during a coronary angiography is depicted in flow chart form in FIG. 2. A balloon-tipped capture catheter can be inserted through the right atrium of the heart and into the coronary sinus (as indicated in step 10). The capture catheter can be configured such that it is capable of withdrawing fluid external to the distal end of the catheter through a flow lumen of the capture catheter. To accomplish this, the capture catheter can include or be connected to a pump or like means capable of creating negative pressure at the distal end to cause fluid to enter the lumen. Once the capture catheter is in place with the distal end within the coronary sinus, the catheter can be connected to, or otherwise placed in signal communication with, one or more automated controllers (step 12) that can control the expansion of a balloon at or near the distal tip of the catheter. The automated controller can also control the timing and flow rate of the contrast removal into the catheter. Contrast medium can then be introduced into at least one of the coronary arteries (step 14) through the use of an insertion catheter or other introduction means. The introduction of the contrast medium can be monitored by the automated controller through electronic communication between the contrast inserting mechanism (e.g., the insertion catheter) and the controller.

After receiving electronic signal input concerning the contrast injection (step 16), which can include, among other things, the timing of the contrast introduction and/or the volume or rate of flow of the injected contrast medium, the controller can then provide an electronic signal to the catheter balloon causing the balloon to inflate and occlude the coronary sinus (steps 18 and 20). Alternatively, the distal end of the catheter can include an integrated sensor (not shown in FIG. 1) that senses the presence of the contrast medium in proximity to the catheter's distal end. The sensor can also be in signal communication with the controller such that when the controller receives a signal from the sensor indicating that contrast medium is present in the coronary sinus (step 22), the controller sends a signal to the catheter balloon to inflate/occlude (step 18), which causes the balloon to inflate and occlude the coronary sinus (step 20). Whether or not the controller includes an integrated sensor, the controller can then send a withdrawal signal to the catheter (step 24) to initiate the withdrawal force (i.e., suction) within the capture catheter. The capture catheter can then drain the blood and contrast medium in the coronary sinus in order to remove a portion of the blood and contrast medium into the catheter (step 26). The controller can then send a signal to the capture catheter 1 to deflate the balloon (step 28), which results in the catheter balloon being deflated (step 30).

A process such as the exemplary process discussed above can be repeated as additional contrast medium is introduced into the coronary arteries, as depicted in FIG. 2 by the arrow from step 30 to step 14. For example, during a coronary angiography there can be several introductions of contrast medium into one or more of the coronary arteries over a period of time, thus requiring several contrast withdrawal phases. Following the withdrawal of the desired amount of contrast medium, the controller can provide a signal to the catheter balloon that causes the balloon to deflate so that the catheter 1 can be removed from the coronary sinus (steps 26, 28, 30 and 34). Prior to removal of the catheter from the coronary sinus (step 34), the presence of contrast medium in the coronary sinus can be checked by fluoroscopy, an integrated sensor positioned near the distal end of the catheter, or other means for determining the presence of contrast medium in the coronary sinus (step 32).

It should be understood that the contrast withdrawal process described above can be modified in numerous ways within the scope of the present invention. For example, in one embodiment the balloon inflation may precede the introduction of the contrast medium into the coronary artery. In another exemplary process of the invention, the capture catheter may have a controller integral with the catheter such that it does not require an additional connectivity step, such as step 12 of FIG. 2. Alternatively, the controller may control some but not all of the controller functions depicted in FIG. 2, such as the balloon inflation/deflation (step 20, 30) but not the contrast withdrawal (step 26).

It should also be understood that in the above described process, in which an automated controller controls the timing of the balloon inflation/deflation and the contrast withdrawal into the catheter, the use of an automated controller is but one of multiple ways to perform this contrast removal process. For example, this process can also be performed manually by an operator in which the initiation of the occlusion (such as the balloon inflation) and the withdrawal of the contrast medium begins before, after, or essentially at the same time as the introduction of contrast medium into one or more of the coronary arteries. In another example of a manual process, manual introduction of contrast medium into a coronary artery is followed by timed manual withdrawal or fluoroscopic observation of the coronary sinus for the presence of contrast medium, which in turn is followed by manual withdrawal of contrast using the capture catheter. This process can also include a measurement of the amount of agent removed following each introduction of contrast medium or other agent into the heart, thereby facilitating a goal of minimizing the amount of contrast medium or other agent left in the body following such a procedure. Quantification of contrast or other agent removed may be accomplished in an automated, “on-line” fashion by use of computerized systems involving detection of both the concentration of contrast or other agent in and amount of fluid removed by the capture device.

The present invention is further illustrated by the following examples, which are not to be construed as limiting the scope of the invention in any manner.

### EXAMPLES

### Example 1

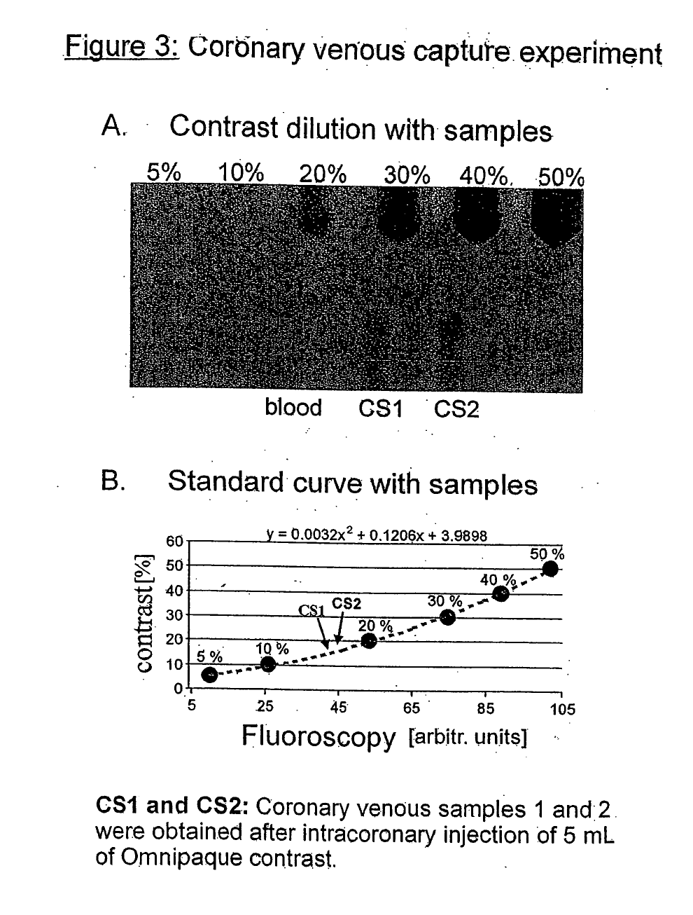

To demonstrate a process of the present invention, coronary angiography was performed in an anesthetized dog in which contrast medium was injected into the left main coronary artery and blood and contrast medium were withdrawn from the coronary sinus. In this example, the catheter balloon was inflated as soon as the contrast injection started. Furthermore, the withdrawal of blood and contrast medium from the coronary sinus was manually controlled and performed under fluoroscopic guidance following injection of the contrast into the left main coronary artery. The collected fluid was analyzed by quantitative fluoroscopy, as exemplified in FIG. 3.

FIG. 3A is a fluoroscopic image showing an array of blood samples containing known concentrations of contrast agent ranging from 0 to 50%. A sample that is 5% contrast agent and 95% blood is denoted 5%. A sample that is 50% contrast agent and 50% blood is denoted 50%. As can be appreciated from this figure, the fluoroscopic density increases with the percentage of contrast agent present. FIG. 3B is a standard curve generated using the data from FIG. 3A. To quantitate recovery of contrast agent, the withdrawn blood was pooled, its volume noted, and its fluoroscopic density determined. The fluoroscopic density was converted to contrast percent using the standard curve. To determine percent recovery, the pooled volume was multiplied by the contrast percent and divided by the injected volume.

In a first trial, 5 milliliters of Iohexol contrast medium (OMNIPAQUE™, Amersham Health, Inc., Princeton, N.J.) were injected into the left main coronary artery of the dog. The catheter balloon was inflated as soon as the contrast injection was started. With the aid of the present invention, 19 milliliters of blood and contrast were recovered from the coronary sinus, of which 15.7% was contrast. This equates to 60% of the injected contrast agent being recovered.

In a second trial, 5 milliliters of contrast medium were again injected into the left main coronary artery. The catheter balloon was inflated as soon as the contrast injection was started. Using an embodiment of the present invention, 26 milliliters of blood and contrast were recovered from the coronary sinus, of which 16.9% was contrast. This equates to 88% of the injected contrast agent being recovered. (See FIG. 3 for representations of the contrast dilution for these trials.)

In a third trial (not depicted in FIG. 3), the catheter balloon was inflated as soon as the contrast injection was started, 10 milliliters of contrast were injected into a coronary artery, and 24.5 milliliters of blood and contrast were recovered from the coronary sinus, of which 26% was contrast. This equates to 63% of the injected contrast agent being recovered from the coronary sinus.

### Example 2

In a follow-on demonstration, it was shown that the electrical conductivity of contrast medium is markedly higher than the electrical conductivity of blood. The electrical resistance of 10 ml of blood was measured (in Ohms), and compared with the separately measured electrical resistance of 10 ml of a contrast medium, Iohexol. With Conductivity=1/Resistance, the difference in conductivity between blood and Iohexol was found to be approximately four-fold. Thus, it was concluded that changes of blood conductivity can potentially be used to detect the presence of contrast in the coronary sinus and trigger a withdrawal pump that is controlled by an automated controller.

### Equivalents

While the invention has been described with reference to various illustrative embodiments and examples, the invention is not limited to the embodiments described. It is evident that many alternatives, modifications and variations of the embodiments described will be apparent to those of ordinary skill in the art. Accordingly, embodiments of the invention as set forth herein are intended to be illustrative, and not limiting the scope of the invention. Various changes can be made without departing from the scope of the invention.

What is claimed is:

1. A method for recovering an agent introduced into a vascular inflow of an organ or tissue, comprising:
inserting a distal end of a catheter into a vascular outflow of an organ or tissue, the catheter comprising a lumen, an outer surface, and an expandable occlusion device secured to the outer surface at the distal end to occlude a flow of fluid through the vascular outflow, while the lumen is in fluid communication with fluid in the vascular outflow;
occluding the flow of fluid through the vascular outflow by expanding the occlusion device; and
withdrawing or passively draining fluid from the vascular outflow through the lumen to recover at least a portion of an agent introduced into a vascular inflow of the organ or tissue.

2. The method of claim 1, wherein the occlusion device is a balloon, and expanding the occlusion device comprises inflating the balloon.

3. The method of claim 1, wherein the agent is a therapeutic or diagnostic agent.

4. The method of claim 1, wherein the agent is a therapeutic agent.

5. The method of claim 4, wherein the therapeutic agent is a viral agent delivered as a vector for gene therapy.

6. The method of claim 1, wherein the agent is a diagnostic agent.

7. The method of claim 6, wherein the diagnostic agent is a contrast medium.

8. The method of claim 1, wherein the withdrawing fluid in the vascular outflow further comprises timing the withdrawing based on a timing of introducing the agent into the vascular inflow of the organ or tissue.

9. The method of claim 8, wherein the withdrawing is controlled by an automated controller in signal communication with the catheter.

10. The method of claim 1, wherein the vascular outflow comprises a coronary sinus.

11. The method of claim 1, wherein the method is performed in conjunction with a coronary angiography.

12. The method of claim 1, wherein the method is performed in conjunction with a non-coronary angiography.

13. The method of claim 1, wherein the withdrawing fluid in the vascular outflow recovers at least 50 percent of the agent introduced into the vascular inflow.

14. The method of claim 1, wherein the vascular inflow comprises a coronary artery.

15. The method of claim 1, further comprising sensing presence of the agent within the vascular outflow.

16. A method of recovering contrast agent during a coronary angiography, comprising:
inserting a catheter into a coronary sinus, the catheter comprising a lumen, an outer surface, a proximal end and a distal end, and an expandable occlusion device secured to the outer surface of the lumen at the distal end to occlude a flow of fluid through a coronary sinus while the lumen is in fluid communication with fluid in the coronary sinus;
introducing a contrast agent into a coronary artery;
expanding the occlusion device to occlude the flow of fluid through the coronary sinus; and
withdrawing fluid from the coronary sinus into the lumen to recover at least a portion of the contrast agent introduced into the coronary artery.

17. The method of claim 16, further comprising sensing a presence of the contrast agent within the coronary sinus.

18. The method of claim 16, further comprising connecting the catheter to an automated device to control rate and timing of the withdrawing of fluid.

19. The method of claim 16, wherein the withdrawing fluid comprises timing of the withdrawing to follow an introduction of the contrast agent into a coronary artery.

20. The method of claim 16, further comprising monitoring an inflow of the contrast agent into the coronary sinus prior to withdrawing fluid from the coronary sinus.

21. The method of claim 16, wherein the withdrawing fluid from the coronary sinus further comprises timing the withdrawing based on a timing of introducing of the contrast agent into the coronary artery.

22. The method of claim 21, wherein the introducing of the contrast agent into the coronary artery triggers the expanding the occlusion device prior to the withdrawing fluid from the coronary sinus.

23. The method of claim 16, wherein the occlusion device is a balloon, and expanding the occlusion device comprises inflating the balloon.

24. The method of claim 23, further comprising deflating the balloon, following the withdrawing fluid from the coronary sinus.

25. The method of claim 24, wherein the deflating the balloon follows a fluoroscopic inspection of the coronary sinus for presence of the contrast agent.

26. The method of claim 24, wherein timing the deflating is based on signal communication from a contrast sensor in the coronary sinus.

27. The method of claim 26, wherein the contrast sensor is integrated with the catheter.

28. An apparatus to capture an agent introduced into a vascular inflow of an organ or tissue, comprising:
a catheter comprising a lumen, an outer surface, a proximal end and a distal end, the distal end being insertable into a vascular outflow of an organ or tissue, and an expandable occlusion device secured to the outer surface at the distal end to occlude a flow of fluid through the vascular outflow;
a pump, connected to the catheter, configured and arranged to withdraw fluid from the vascular outflow into the lumen; and
an automated controller in signal communication with the catheter to control the expandable occlusion device to occlude the flow of fluid through the vascular outflow, and to control the pump to withdraw at least a portion of the agent from the vascular outflow, the controlling of the occlusion device and the pump based on at least the timing of the agent administered into the vascular inflow.

29. The apparatus of claim 28, further comprising a sensor integrated with the catheter and in signal communication with the expandable occlusion device and the pump, to detect presence of the agent in the vascular outflow and to communicate the detection to at least one of the occlusion device and the pump.

30. The apparatus of claim 29, wherein the integrated sensor comprises at least one electrode disposed at the distal end of the catheter to detect presence of the agent in the vascular outflow.

31. The apparatus of claim 28, wherein the expandable occlusion device is an inflatable balloon.

32. The apparatus of claim 28, wherein the agent is a diagnostic agent.

33. The apparatus of claim 32, wherein the diagnostic agent is a contrast medium.

34. The apparatus of claim 28, wherein the agent is a therapeutic agent.

35. The apparatus of claim 34, wherein the therapeutic agent is a viral agent delivered as a vector for gene therapy.

36. The apparatus of claim 28, wherein the vascular outflow is a coronary sinus.

37. The apparatus of claim 28, wherein the vascular inflow comprises a coronary artery.

38. The apparatus of claim 28, wherein the lumen has a diameter within the range of from approximately 10 micrometers to approximately 4 centimeters.

39. A device to capture a contrast agent introduced into a coronary artery during a coronary angiography, comprising:
a catheter comprising a lumen, an outer surface, a proximal end and a distal end, the distal end being insertable into a coronary sinus, and an inflatable balloon secured to the outer surface at the distal end to occlude a flow of fluid in the coronary sinus;
a pump, operably coupled to the catheter, configured and arranged to withdraw fluid from the coronary sinus into the lumen; and
an automated controller in signal communication with the catheter to control the inflatable balloon to occlude the flow of fluid in the coronary sinus, and to control the pump to withdraw at least a portion of the agent from the vascular outflow, the controlling of the occlusion device and the pump based on timing and rate of introduction of an agent introduced into the vascular inflow.

40. The device of claim 39, further comprising a sensor integrated with the catheter in proximity to the distal end and in signal communication with the inflatable balloon and the pump, to detect presence of contrast agent in the coronary sinus and to communicate the detection to at least one of the inflatable balloon and the pump.
